(19) United States
(12) Patent Application Publication
(10) Pub. No.: US 2023/0218679 A1
(43) Pub. Date: Jul. 13, 2023

(54) COMPOSITION AND METHOD FOR OMEGA-3 AND PLANT FLAVONOIDS FOR INCREASING HDL CHOLESTEROL AND DECREASING LDL CHOLESTEROL AND TRIGLYCERIDE LEVELS

(71) Applicant: Viva Life Science, Inc., Costa Mesa, CA (US)

(72) Inventor: Thuong Cao Do, Costa Mesa, CA (US)

(21) Appl. No.: 18/095,468

(22) Filed: Jan. 10, 2023

Related U.S. Application Data
(60) Provisional application No. 63/298,100, filed on Jan. 10, 2022.

(51) Int. Cl. A61K 35/60; A61K 9/00; A61K 36/55; A61K 31/255; A61K 31/202; A61K 36/48; A61K 36/8962; A61K 36/752; A61K 45/06; A61K 31/352

(52) U.S. Cl. CPC A61K 35/60; A61K 9/0053; A61K 36/55; A61K 31/255; A61K 31/202; A61K 36/48; A61K 36/8962; A61K 36/752; A61K 45/06; A61K 31/352

(57) ABSTRACT

The present invention relates to nutritional supplements for the human diet used to manipulate HDL cholesterol, LDL cholesterol, and triglyceride levels in human blood plasma. The invention contains a novel combination of eicosapentaenoic acid (EPA) and docosahexaenoic acid (DHA) present in Fish Oil, alpha-linolenic acid (ALA) present in Flaxseed Oil, Vitamin E in the form of d-alpha-tocopherol, Allicin present in Garlic, Phosphatidylcholine present in Lecithin, plant phytochemicals present in Parsley herb, citrus flavonoids present in Lemon fruit and Rutin (from Sophora japonica), and Capsaicin present in Cayenne Pepper.

### FIELD OF THE INVENTION

The present invention relates to nutritional supplements for the human diet used to promote favorable changes to HDL cholesterol, LDL cholesterol, and triglyceride levels in human blood plasma.

### BACKGROUND OF THE INVENTION

As of May 2017, the World Health Organization has reported that cardiovascular diseases (CVDs) are the number one cause of death globally. In 2016 alone, it is estimated that 17.6 million people died of CVDs representing 31% of all global deaths. Of these 17.6 million people who died of CVDs, 85% were due to heart attack and stroke (World Health Organization. Cardiovascular Diseases (CVDs). The United States, it is estimated that 610,000 people die of heart disease every year, representing 25% of all deaths (Center for Disease Control and Prevention. Heart Disease Facts. Most CVDs can be prevented by abstaining from tobacco use, leading a healthy lifestyle by being active and having a nutritious diet. People who are at high cardiovascular risk may have risk factors such as hypertension, hyperlipidemia or diabetes. Studies have shown that there is an increased risk of coronary heart disease and stroke in adults with high triglyceride levels and low HDL levels (Jennifer S. Lee, Po-Yin Chang, Ying Zhang, Jorge R. Kizer, Lyle G. Best and Barbara V. Howard, “Triglyceride and HDL-C Dyslipidemia and Risks of Coronary Heart Disease and Ischemic Stroke by Glycemic Dysregulation Status: The Strong Heart Study” and K. E. L Harchaoui, M. E Visser, J. J. P Kastelein, E. S Stroes, and G. M Dallinga-Thie. “Triglycerides and Cardiovascular Risk). Studies have shown that a diet high in healthy fats such as Omega-3 fatty acids from marine and plant sources provide positive benefits for cardiovascular health”; JoAnn E. Manson, M.D., Dr.P.H., Nancy R. Cook, Sc.D., I-Min Lee, M.B., B.S., Sc.D., William Christen, Sc.D., Shari S. Bassuk, Sc.D., Samia Mora, M.D., M.H.S., Heike Gibson, Ph.D., Christine M. Albert, M.D., M.P.H., David Gordon, M.A.T., Trisha Copeland, M.S., R.D., Denise D'Agostino, B.S., Georgina Friedenberg, M.P.H., et al., for the VITAL Research Group, “Marine n-3 Fatty Acids and Prevention of Cardiovascular Disease and Cancer”). These studies have also extended to the use of plant flavonoids that also have positive benefits for cardiovascular health. With this information, the invention of this nutritional supplement containing Omega-3 fatty acids and plant flavonoids may benefit cardiovascular health by manipulating levels of HDL cholesterol, LDL cholesterol, and triglycerides (Suvi Manninen, Maria Lankinen, Arja Erkkilä,Su Duy Nguyen, Maija Ruuth. Vanessade Mello, Katariina Öörni, Ursula Schwab “The effect of intakes of fish and Camelina sativa oil on atherogenic and anti-atherogenic functions of LDL and HDL particles: A randomized controlled trial” and Wendy A. M. Blom, Wieneke P. Koppenol, Harry Hiemstra, Tatjana Stojakovic, Hubert Scharnagl, Elke A. Trautwein “A low-fat spread with added plant sterols and fish omega-3 fatty acids lowers serum triglyceride and LDL-cholesterol concentrations in individuals with modest hypercholesterolaemia and hypertriglyceridaemia”) to accepted recommended levels as seen in Table 1 (NIH. U.S. National Library of Medicine. MedlinePlus. “Cholesterol Levels: What You Need to Know,” which may lead to lower incidences of cardiovascular disease (R. Preston Mason “New Insights into Mechanisms of Action for Omega-3 Fatty Acids in Atherothrombotic Cardiovascular Disease”).

TABLE 1
Table 1: Healthy Cholesterol Levels Based
on Age and Gender (Reference: Medline Plus)

| | |
|---|---|
| Type of Cholesterol | Healthy Level |

Anyone age 19 or younger:

| | |
|---|---|
| Total Cholesterol | Less than 170 mg/dL |
| Non-HDL | Less than 120 mg/dL |
| LDL | Less than 100 mg/dL |
| HDL | More than 45 mg/dL |

Men age 20 or older:

| | |
|---|---|
| Total Cholesterol | 125 to 200 mg/dL |
| Non-HDL | Less than 130 mg/dL |
| LDL | Less than 100 mg/dL |
| HDL | 40 mg/dL or higher |

Women age 20 or older:

| | |
|---|---|
| Total Cholesterol | 125 to 200 mg/dL |
| Non-HDL | Less than 130 mg/dL |
| LDL | Less than 100 mg/dL |
| HDL | 50 mg/dL or higher |

TABLE 2
Table 2: Healthy Triglyceride levels (reference: MedinePlus)

| Category | Triglyceride Level |
|---|---|
| Normal | Less than 150 mg/dL |
| Borderline high | 150 to 199 mg/dL |
| Very high | 500 mg/dL and above |

The three main Omega-3 fatty acids are alpha-linolenic acid (ALA), eicosapentaenoic acid (EPA) and docosahexaenoic acid (DHA). ALA can be found in plants such as flaxseed, chia seed and soybean. EPA and DHA can be found in algae, fish and other marine sources. ALA is an essential fatty acid that must be consumed from plant or animal sources. ALA can be converted in the body to EPA, then EPA to DHA, but only in small amounts. The best way to increase levels of Omega-3′s in the body is to consume Omega-3 rich foods or by taking nutritional supplements. Omega-3′s are important components of cell membranes and have supportive functions in the heart and blood vessels. Many studies have shown that eating fatty fish may help protect you from heart issues and that getting high levels of EPA and DHA may also lower triglyceride levels in adults (Omega-3 Fatty Acids. National Institute of Health—Office of Dietary Supplements.

Flavonoids have been shown to have antioxidant, anti-inflammatory and vasodilator properties that may lead to positive cardiovascular benefits (Julia J. Peterson, Johanna T. Dwyer, Paul F. Jacques, and Marjorie L. McCullough “Do Flavonoids Reduce Cardiovascular Disease Incidence or Mortality in US and European Populations?”; R. D. Mendonca, N. C. Carvalho, J. M. Martin-Moreno, A. M. Pimenta, A. C. S. Lopes, A. Gea, M. A. Martinez-Gonzalez, M. Bes-Rastrollo “Total polyphenol intake, polyphenol subtypes and incidence of cardiovascular disease: The SUN cohort study”; Nicola P. Bondonno, Frederik Dalgaard, Cecilie Kyr∅, Kevin Murray, Catherine P. Bondonno, Joshua R. Lewis, Kevin D. Croft, Gunnar Gislason, Augustin Scalbert, Aedin Cassidy, Anne Tjønneland, Kim Overvad & Jonathan M. Hodgson “Flavonoid intake is associated with lower mortality in the Danish Diet Cancer and Health Cohort”; Dina Maaliki, Abdullah A. Shaito, Gianfranco Pintus, Ahmed El-Yazbi, Ali Heid “Flavonoids in hypertension: a brief review of the underlying mechanisms”; Taylor Wallace, Aedin Cassidy, Mei Chung, Mario Ferruzzi, Paul Jacques, Elizabeth Johnson, Ding Wang, Naisi Zhao, Marissa Shams-White, Micaela Karlsen “Tea Flavonoids and Risk of Cardiovascular and All-Cause Mortality: A Systematic Review and Meta-Analysis” (P06-126-19); and Nicola P. Bondonno, Joshua R. Lewis, Lauren C. Blekkenhorst, Catherine P. Bondonno, John H. C. Shin, Kevin D. Croft, Richard J. Woodman, Germaine Wong, Wai H. Lim, Bamini Gopinath, Victoria M. Flood, Joanna Russell, Paul Mitchell, Jonathan, M. Hodgson “Association of flavonoids and flavonoid-rich foods with all-cause mortality: The Blue Mountains Eye Study”). Garlic is one such example to have these cholesterol lowering effects. Studies through the years have shown that bioactive compounds in garlic, such as allicin, have shown in human and animal studies to have cholesterol lowering effects (N. C. Morcos, MD, PhD “Modulation of Lipid Profile by Fish Oil and Garlic Combination” N.C. Journal of the National Medical Association, Volume 89, Number 10 and Ellen Tattelman, M.D. “Health Effects of Garlic,” Albert Einstein College of Medicine of Yeshiva University, Bronx. N.Y.). Capsaicin, a capsaicinoid found in chili peppers, influences the modulation of lipids and effectively reduces cholesterol and triglyceride levels (Musunuru Suneel Kumar Reddy and Uma V. Manjappara “Capsaicin but not Genistein Influences Modulation of Lipid Parameters by Obestatin in DIO-057BL/6 Mice”). Rutin, a citrus flavonoid, found in citrus fruits, as well as in many other plants, plays a role in the reduction of total triglyceride levels and has antioxidative properties (Aditya Ganeshpurkar and Ajay K. Saluja “The Pharmacological Potential of Rutin” and Lin Lv, Yucai Yao, Gang Zhao and Guiyue Zhu “Rutin inhibits coronary heart disease through ERK1/2 and Akt signaling in a porcine model”). Vitamin E has antioxidative and cardio-protective properties. Ingestion of Vitamin E rich foods has been associated with lower risk of cardiovascular disease (Joshua B. Wiener, Elliot M. Berinstein, Andrew P. Levy “Clinical Cardiovascular Disease Trials: The Vitamin E Case”). Phosphatidylcholine found in soybean, is made up of phospholipids and is a major component of cell membranes. Studies have linked phosphatidylcholine to homocysteine, a homologue of the amino acid cysteine, concluding that there is a correlation of higher phosphatidylcholine intake and lower homocysteine leading to lower cardiovascular disease (Michele F. Panunzio, Alessandra Pisano, Antonietta Antoniciello, Vincenzo Di Martino, Lucia Frisoli, Vincenzo Cipriani, Maria A. Mongelli, Giorgio Bronzetti “Supplementation with fruit and vegetable concentrate decreases plasma homocysteine levels in a dietary controlled trial” and Samir Samman, Gayathri Sivarajah, June C. Man, Ziaul I. Ahmad, Peter Petocz, Ian D. Caterson “A Mixed Fruit and Vegetable Concentrate Increases Plasma Antioxidant Vitamins and Folate and Lowers Plasma Homocysteine in Men”). Parsley has been associated with heart health for many years now. Parsley contains B vitamins such as folic acid and contains antioxidative properties. Like phosphatidylcholine, there is a link between parsley consumption with lower homocysteine levels, in turn leading to lower cardiovascular disease (Akira Kawashima, MD, PhD; Takeo Madarame, MD, PhD; Hiroto Koike, MD, PhD; Yasuhiro Komatsu, PhD and John A Wise, PhD “Four week supplementation with mixed fruit and vegetable juice concentrates increased protective serum antioxidants and folate and decreased plasma homocysteine in Japanese subjects”). Green tea contains polyphenols and catechins that may also lower LDL and total cholesterol. Studies have also shown that drinking green tea may help lower systolic and diastolic blood pressure.

Soy isoflavones found in legumes such as edamame may help lower total cholesterol and LDL cholesterol. Lycopene such as is found in tomatoes and other red vegetables and fruits such as red carrots, papayas, gac melons, and watermelons have heart healthy properties that may reduce the risk of stroke and heart disease. Eating lycopene rich foods may also help increase HDL cholesterol which may lead to the removal of excess cholesterol and plaque that may harden and narrow arteries.

### SUMMARY OF THE INVENTION

The invention is a nutritional supplement and is used to supplement deficiencies in the diet as well as provide beneficial cardiovascular effects. By combining Fish Oil, Flaxseed Oil, Vitamin E, Lecithin, Garlic, Lemon, Parsley, Rutin, Cayenne pepper, Green Tea, Soy Isoflavones, and Lycopene in the form of a liquid encapsulated gelatin shell, this composition may be orally administered as directed to obtain nutritional and cardiovascular benefits. These cardiovascular benefits may include the lowering of LDL cholesterol and triglycerides while increasing HDL cholesterol to healthy levels. Compositions of the invention include: 1) combinations comprising EPA and DHA from Fish Oil, ALA from Flaxseed Oil, Vitamin E, Lecithin, Garlic, Lemon, Parsley, Rutin, Cayenne pepper, Green Tea, Soy Isoflavones, and Lycopene and combinations thereof; and 2) combinations comprising EPA and DHA from Fish Oil, Vitamin E, Lecithin, Garlic, Lemon, Parsley, Rutin and Cayenne pepper.

### DETAILED DESCRIPTION OF THE INVENTION

The first example of the invention is comprised of two parts, (1) a liquid composed of Fish Oil, Flaxseed Oil, Isoflavones, garlic containing Allicin, and Flavonoids, and optionally an additional composition or compositions selected from the group consisting of Vitamin E, Lecithin, Lemon, Parsley, Rutin, Cayenne pepper, Green Tea, and Lycopene (2) a flexible outer layer encapsulating the liquid composed of Gelatin, Glycerin and Water. This combination of a liquid inner portion and flexible outer layer will be referred to as a softgel. The liquid inner portion may contain additional plant ingredients and is not limited to the present ingredients listed.

(1) The liquid inner portion is comprised of:
1. 500 mg to 3000 mg of Fish Oil containing 100 mg to 1000 mg of EPA and 50 mg to 500 mg of DHA, including but not limited to a range between 2300 mg to 2500 mg of Fish Oil containing 650 mg to 850 mg of EPA and 450 mg to 650 mg DHA
2. 100 to 500 mg of Flaxseed Oil containing 22.5 mg to 112.5 mg of ALA, including but not limited to composition containing 200 mg 300 mg of Flaxseed oil containing 45 mg to 67.5 mg of ALA.
3. 4 IU to 30 IU of Vitamin E, including but not limited to 18 international units to 22 international units of Vitamin D as alpha-tocopherol.
4. 2 mg to 20 mg of Lecithin containing Phosphatidylcholine, including but not limited to 8 mg to 12 mg of lecithin containing phosphatidylcholine.
5. 150 mg to 1000 mg of Garlic containing allicin, including but not limited to 650 mg to 850 mg of Garlic containing allicin.
6. 20 mg to 100 mg of Lemon containing bioflavonoids, including but not limited to 80 mg to 100 mg of Lemon containing bioflavonoids.
7. 20 mg to 200 mg of Parsley containing flavonoids, including but not limited to 150 mg to 170 mg of Parsley containing flavonoids.
8. 10 mg to 100 mg of Rutin, preferably from Sophora japonica, including but not limited to 45 mg 65 mg of Rutin from Sophora japonica.
9. 10 mg to 100 mg of Cayenne pepper containing capsaicin, including but not limited to 50 mg to 70 mg of Cayenne pepper containing capsaicin.
10. 10 mg to 100 mg of Green Tea containing polyphenols, including but not limited to 75 mg to 95 mg of Green Tea containing polyphenols
11. 10 mg to 100 mg of Isoflavones including selected compositions of isoflavones extracted from soybeans or extracts from other species so long as the extracted species contain certain isoflavone compositions as follows

10 mg to 100 mg of Lycopene, preferably from tomatoes or other plant species including 70 mg to 90 mg of lycopene from tomatoes and selected groups selected individually from the compositions described above and combinations thereof as described below.

(2) The flexible outer layer of the gel caps is preferably comprised of:
1. 300 mg to 500 mg of Gelatin.
2. 100 mg to 300 mg of Glycerin.
3. 200 mg to 400 mg of Water.

A single softgel will be taken orally and will contain 1030 mg to 1500 mg of liquid and 600 mg to 1200 mg of the outer layer. The dosage will range from one to six softgels, preferably 4 softgel capsules per day. The overall dimension of the softgel is approximately 25 mm by 10 mm by 10 mm.

The second example of the invention is comprised of two parts, (1) a liquid composed of Fish Oil, Vitamin E, Lecithin, Garlic, Lemon, Parsley, Rutin and Cayenne pepper, (2) a flexible outer layer encapsulating the liquid composed of Gelatin, Glycerin and Water. This combination of a liquid inner portion and flexible outer layer will be referred to as a softgel. The liquid inner portion may contain additional plant ingredients and is not limited to the present ingredients listed.

(1) The liquid inner portion is comprised of:
1. 500 mg to 3000 mg of Fish Oil containing 100 mg to 1000 mg of EPA and 50 mg to 500 mg of DHA.
2. 4 IU to 30 IU of Vitamin E as d-alpha-tocopherol.
3. 2 mg to 20 mg of Lecithin containing Phosphatidylcholine.
4. 150 mg to 1000 mg of Garlic containing allicin.
5. 20 mg to 100 mg of Lemon containing bioflavonoids
6. 20 mg to 200 mg of Parsley containing flavonoids.
7. 10 mg to 100 mg of Rutin from Sophora japonica.
8. 10 mg to 100 mg of Cayenne pepper containing capsaicin.

(2) The flexible outer layer is comprised of:
1. 300 mg to 500 mg of Gelatin.
2. 100 mg to 300 mg of Glycerin.
3. 200 mg to 400 mg of Water.

A single softgel will be taken orally and will contain 1000 mg to 1200 mg of liquid and 600 mg to 1200 mg of the outer layer. The dosage will range from one to six softgels, preferably 4 softgel capsules per day. The overall dimension of the softgel is approximately 25 mm by 10 mm by 10 mm. The manufacturing of the invention is performed as follows: The liquid inner portion of the mixture is combined in a mixing vessel in the order listed. Once the mixture is thoroughly mixed and homogenous, the mixture is set aside for the encapsulation step. The outer layer is produced as follows: water is added to a heated mixing vessel and brought to a temperature between 50° C. to 60° C., glycerin then be added to the mixing vessel. The temperature is maintained between 50° C. to 60° C., gelatin is than added and then mixed until the mixture is homogenous. The outer layer mixture, also referred to as the gel mass, is added to the encapsulation machine. The inner composition, also referred to as the “fill,” is then be added to the encapsulation machine. The next step is the encapsulation process wherein the gel mass is wrapped around the core composition combination comprising the fill to create a softgel. Once the softgel is produced, the assembly is dried until the outer layer does not contain more than 5% water content. The softgel are then be sorted, inspected and cleaned. Once the softgel is cleaned, a suitable plurality of the softgels consistent with a specified number of oral dosages are than packaged and ready for human consumption. The encapsulation machine is commercially available and many companies provide services to produce softgels. One such company is CAP PLUS TECHNOLOGIES (CAP PLUS TECHNOLOGIES). A visual representation of how a softgel is produced can be found here www.capplustech.com/how to softgel.htm

Each of these ingredients listed in the two examples are commercially available from many suppliers. Fish oil containing EPA and DHA can be sourced from fishes such as mackerel (Scomber scombrus), sardines (Sardina pilchardus), and menhaden (Brevoortia tyrannus). The process in which fish oil is produced from these fishes involve the steam rendering of the fresh fish, sludge decanter to separate the oil and water from the dry matter, vacuum drying, deacidification, vacuum drying a second time. This dried mass is then filtered of high melting components, producing crude fish oil, which is then deodorized to remove odors. Natural absorbents are used to remove polychlorinated biphenyl and pesticides, and an analysis is performed. The mass then goes through a crystallization process, homogenizing and blending of the fish oil, and an analysis is performed., then through filtration and packaging. Flaxseed oil containing ALA can be sourced from Flax (Linum usitatissimum). Flaxseed oil is produced as follows: mechanically cleaning of flax, pressing of flax, collection of oily fraction, analysis performed , then filtration and packaging. Vitamin E is produced from soybean (Glycine max). It is produced by starting with soybean oil, esterification with methanol and sulfuric acid, producing a crystal liquid, which is then refrigerated. The liquid is then filtered and distilled, producing crude mixed tocopherols. Column chromatography is performed separating the mixed tocopherols, solvents are then removed, the crude alpha tocopherols are produced, and then distillation of the crude alpha tocopherols refines the d-alpha-tocopherol. Lecithin is produced from soybeans (Glycine max) by first starting with crude soybean oil, degumming is performed to produce crude soybean lecithin, de-oiling the crude soybean lecithin produces oil-free lecithin, then concentrating the lecithin, drying and packaging. Garlic (Allium sativum) used in this invention is produced by first extracting with water into concentrated garlic extract. The garlic extract is then spray-dried and ground to a powder. An analysis of allicin is performed and then the garlic powder is packaged. Lemon (Citrus limon) fruit containing bioflavonoids is produced by removing the juice and oil, then the lemon is milled to the ideal extraction size. Bioflavonoids are extracted and concentrated with water.

The bioflavonoid concentrate is dried, ground to a powder , and an analysis of bioflavonoids is performed, then packaged. Parsley (Petroselinum crispum) herb powder is produced by first cleaning the parsley, then dehydrating and milling to a powder. A microbial analysis is performed and then the parsley powder is packaged. Rutin is produced from Sophora japonica flower buds. The flower buds are milled to a powder , and then soaked in 100° C. water for extraction. The soaked flower buds powder are filtered, the liquid is then crystallized using hydrochloric acid, and the crystallized material is then filtered and dried, then milled into a powder. An analysis of rutin content is performed and then the rutin is packaged. Cayenne Pepper (Capsicum frutescens) powder is produced by first cleaning the peppers, then dehydrating the peppers. Once the peppers are dried they are milled into a powder , then the powdered cayenne pepper is analyzed for capsaicin content, and then packaged. Green Tea (Camellia sinensis) is produced by first separating the leaves from the stems. The green tea leaves are then soaked in water to remove the polyphenols from the tea leaves. The tea leaves are filtered leaving behind the polyphenols. The polyphenols are concentrated then spray dried. The concentrated green tea polyphenols are then blended, deionized and packaged.

Soy Isoflavones are produced by first washing the soybeans (Glycine max), then extracting the isoflavones by soaking the soybeans with water and ethanol. The soybeans are filtered leaving behind the isoflavones. The isoflavones are further concentrated by removing additional liquid then it is spray dried. The concentrated isoflavones are then grinded, sifted and then packaged. Lycopene is produced by separating the tomato (Lycopersicum esculentum) peel from the fruit. The lycopene is extracted from the peel producing a lycopene oleoresin. The lycopene oleoresin is filter, crystallized and then dried. The lycopene crystal is then grinded, sifted and packaged.

Those of ordinary skill in the art will readily recognize, in light of this description, the many variations of suitable formulation and manufacturing procedures are suitable to fabricate the nutraceutical compositions described herein.

As used herein and in the appended claims, the singular forms “a”, “an”, and “the” include plural referents unless the context clearly dictates otherwise.

Where a range of values is provided, each intervening value, to the tenth of the unit of the lower limit unless the context clearly dictates otherwise, between the upper and lower limit of that range and any other stated or intervening value in that stated range, is encompassed within the disclosure and can be claimed as a sole value or as a smaller range. Where the stated range includes one or both of the limits, ranges excluding either or both of those included limits are also included in the disclosure.

Where a discrete value or range of values is provided, that value or range of values may be claimed more broadly than as a discrete number or range of numbers, unless indicated otherwise. For example, each value or range of values provided herein may be claimed as an approximation and this paragraph serves as antecedent basis and written support for the introduction of claims, at any time, that recite each such value or range of values as “approximately” that value, “approximately” that range of values, “about” that value, and/or “about” that range of values. Conversely, if a value or range of values is stated as an approximation or generalization, e.g., approximately X or about X, then that value or range of values can be claimed discretely without using such a broadening term.

However, in no way should this specification be interpreted as implying that the subject matter disclosed herein is limited to a particular value or range of values absent explicit recitation of that value or range of values in the claims. Values and ranges of values are provided herein merely as examples.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

I claim:

1. A method of increasing HDL cholesterol and lowering LDL cholesterol and also lowering total triglyceride levels to healthy ranges in the human blood plasma by orally administering a nutritional supplementing amount of a combination of compositions comprising:
Fish Oil, Flaxseed Oil, Isoflavones, Allicin, bioflavonoids and combinations thereof.

2. The method of claim 1, wherein the fish oil composition comprises between 500 mg and 3000 mg of Fish Oil containing 100 mg to 1000 mg of EPA and 50 mg to 500 Mg of DHA.

3. The method of claim 1, wherein the Flaxseed Oil composition comprises between 100 to 500 mg of Flaxseed Oil containing 22.5 mg to 112.5 mg of ALA.

4. The method of claim 1, wherein the isoflavones composition comprises between 10 mg to 100 mg of Isoflavones.

5. The method of claim 4, wherein the isoflavones composition is derived from soybeans.

6. The method of claim 4, wherein the allicin composition comprises between 150 mg to 1000 mg of garlic containing the allicin.

7. The method of claim 1, wherein the bioflavonoids composition is further comprised of 200 mg to 100 mg of lemon containing the bioflavonoids.

8. The method of claim 1, further comprising orally administering 4 IU to 30 IU of Vitamin E.

9. The method of claim 8, wherein the vitamin E composition is d-alpha-tocopherol.

10. The method of claim 1, further comprising orally administering a composition selected from the group consisting of phosphatidylcholine, flavonoids, Rutin, capsaicin, polyphenols, Lycopene and combinations thereof.

11. A dietary composition comprising:
an encapsulated liquid having a combination of compositions comprising:
Fish Oil, Flaxseed Oil, Isoflavones, Garlic containing Allicin, bioflavonoids and combinations thereof.

12. The dietary composition of claim 11 wherein the Fish Oil composition comprises between 500 to 3000 mg of Fish Oil containing 100 mg to 1000 mg of EPA and 50 mg to 500 mg of DHA.

13. The dietary composition of claim 11 wherein the Flaxseed Oil composition is comprised of between 100 to 500 mg of Flaxseed Oil containing 22.5 mg to 112.5 mg of ALA.

14. The dietary composition of claim 11 wherein the isoflavones composition is comprised of between 10 mg to 100 mg of Isoflavones.

15. The dietary composition of claim 12 wherein the isoflavones are derived from soybeans.

16. The dietary composition of claim 11, wherein the garlic composition is between 150 mg and 1000 milligrams.

17. The dietary composition of claim 11 wherein the bioflavonoids composition comprises 20 mg to 100 mg.

18. The dietary composition of claim 17, wherein the bioflavonoids are derived from Lemon.

19. The dietary composition of claim 11, further comprising 4 IU to 30 IU of Vitamin E.

20. The dietary composition of claim 19, wherein the Vitamin E is d-alpha-tocopherol.

21. The dietary composition of claim 11 further a composition selected from the group consisting of comprising 2 mg to 20 mg of Lecithin containing Phosphatidylcholine, 20 mg to 200 mg of Parsley containing flavonoids, 10 mg to 100 mg of Rutin, 10 mg to 100 mg of Cayenne pepper containing capsaicin, 10 mg to 100 mg of Green Tea containing polyphenols, 10 mg to 100 mg of Lycopene.
